(19) United States
(12) Patent Application Publication
(10) Pub. No.: US 2007/0178062 A1
(43) Pub. Date: Aug. 2, 2007

(54) Treatment of presbyopia with picolinic acid and its analogs

(76) Inventors: Nathan Ravi, St. Louis, MO (US); Paul D. Hamilton, St. Louis, MO (US)

Correspondence Address: BLANK ROME LLP, 600 NEW HAMPSHIRE AVENUE, N.W., WASHINGTON, DC, 20037, US

(21) Appl. No.: 11/544,647

(22) Filed: Oct. 10, 2006

Related U.S. Application Data
(60) Provisional application No. 60/724,305, filed on Oct. 7, 2005.

(51) Int. Cl. A61K 31/785; A61K 31/58; A61K 31/455; A61K 31/335

(52) U.S. Cl. 424/78.27; 424/425; 424/427; 514/176; 514/355; 514/450

(57) ABSTRACT

Compositions and methods for inhibiting the growth of lens epithelial cells are provided. The compositions provided include a chelating agent in an amount sufficient for inhibiting the growth of lens epithelial cells. The compositions and methods provided may also be used for the treatment of disorders of the eye, especially in the treatment of presbyopia.

### REFERENCE TO RELATED APPLICATION

The present application claims the benefit of U.S. Provisional Patent Application No. 60/724,305 filed Oct. 7, 2005, which disclosure is hereby incorporated by reference in its entirety into the present application.

### FIELD OF THE INVENTION

The present invention relates to topical compositions and methods for treatment of presbyopia and other disorders of the eye.

### BACKGROUND OF THE INVENTION

The human lens is the only intraocular tissue that continues to grow throughout life, albeit at decreasing rates with advancing age (1;2). Some researchers have associated this progressive growth to cause presbyopia (3;4) and posterior capsular opacification (PCO)(5-8). Presbyopia is a clinical condition in which the speed and amplitude of accommodation of a normal eye decreases with advancing age (9) (10). On the other hand, PCO occurs only in patients who have undergone cataract surgery in which the posterior capsule is left intact.

The pathogenesis of presbyopia is clearly multifactorial (11-14); however, continually increasing mass as well as size of the lens is hypothesized to alter the force vectors generated by the ciliary body during the focusing process (4;15). Currently, there is no medical treatment for presbyopia except for external prosthesis. Bifocals or monocular contact lenses, are at best, inadequate treatments for this condition and provide clear vision at only two distances. Presbyopia occurs in almost everyone by fifty years of age, irrespective of race, sex, geographic location, diet, or economical status (16-18). Its economic cost from denial, treatment, management, and lost productivity is conservatively estimated to be at least ten billion dollars per year in the US alone. Seleral expansion (19;20) and improved design in interocular lenses (IOL) (21-24) are two of the surgical treatments being evaluated for this condition. Both have met with some subjective improvement in accommodation. A truly injectable accommodating lens formed by refilling the pre-evacuated capsular bag for the preservation of accommodation after cataract surgery is also an active area of research (25-28).

PCO continues to be the most common complication of intra-ocular lens implant surgery despite recent important advancements in implant design and surgical techniques (29). Generally, as many as 35% of patients begin to experience decreased visual acuity within two years of undergoing lens implant surgery. During cataract surgery it is almost impossible to remove the entire cells adherent to the anterior and equatorial regions of the lens capsule. These remnant epithelial cells continue to proliferate and migrate on the lens capsule (30-33). The epithelial cell's effort to refill the capsular bag is marred by disorganized growth pattern and wrinkling of the posterior capsules, which tend to scatter light and consequently deteriorate visual acuity. Once symptomatic, the patient's vision can be restored only by excising the PCO particularly along the visual axis. Laser photo-disruption of the posterior capsule with the high-energy neodymium:yttrium-aluminum-garnet (Nd:YAG) laser is the preferred method. This procedure, although technically simple is not innocuous, and retinal detachment (34;35) is one of its potentially damaging side effects. Several pharmaceutical agents inhibit PCO in vitro or in animal models. For example, ethylenediaminetetraacetic acid (EDTA) (36;37), methotrexate (38), mitomycin (39-42), daunomycin (43-46), 5-fluorouracil (46;47), daunorubicin (44;48-50), colchicine (51;52), and cacimycin (53), have all been found to prevent the proliferation of lens epithelial cells in-vitro. Unfortunately, all of the above agents lack specificity and have a narrow therapeutic index. To circumvent non-specificity, lens epithelial cell specific antibodies conjugated with toxins have also been investigated (54-56). Clinical trials using these intra-operative techniques seem promising (57;58).

There is a need in the art for compounds capable of inhibiting the growth of the lens epithelial cells as a medical or surgical treatment for presbyopia. Identifying a non-toxic natural metabolite that could act specifically or preferentially to inhibit growth of lens epithelial cells when administered topically could be useful in slowing the onset of presbyopia as well as aiding in the treatment of PCO. Such an agent could also play a pivotal role in the surgical treatment of presbyopia in which the almost intact capsular bag is refilled with a pliable material whose refractive power could be molded by the lens capsular membrane. This pliable material could then potentially auto-focus and would restore some accommodation after cataract surgery. To achieve this, the material not only needs to be injectable through a small capsulorhexisis and be pliable enough to be molded by the capsular pressure but also be able to totally inhibit PCO as photo-disruption of the capsular bag would render the implant dysfunctional.

### SUMMARY OF THE INVENTION

In order to meet the need in the art described above, the present invention provides compositions capable of inhibiting the growth of lens epithelial cells and further capable of treating or preventing a disorder of the eye.

It is an object of the present invention to provide compositions for inhibiting the growth of lens epithelial cells. The compositions of the invention contain a chelating agent in an amount sufficient to inhibit lens epithelial cell growth. In specific embodiments of compositions of the invention, the chelating agent may be picolinic acid, fusaric acid, di-piconlinic acid or nicotinic acid.

It is a further object of the present invention to provide pharmaceutical compositions for topical application to the eye containing a chelating agent in an amount capable of inhibiting lens epithelial cell growth and one or more excipients. The pharmaceutical compositions of the present invention may be used for treating or preventing a disorder of the eye. In one embodiment, the pharmaceutical compositions of the invention are used for treating or preventing presbyopia.

It is a still further object of the invention to provide methods for inhibiting the growth of lens epithelial cells. The methods of the present invention involve contacting lens epithelial cells with an amount of a chelating agent sufficient to inhibit their growth.

It is a still further object of the invention to provide methods for treating or preventing a disorder of the eye. The methods of the present invention involve topically applying to an eye a composition including a chelating agent in an amount sufficient to inhibit the growth of lens epithelial cells and one or more excipients.

### BRIEF DESCRIPTION OF THE DRAWINGS

Various experimentally verified uses for a preferred embodiment are disclosed in detail with respect to the drawings, as listed.

FIG. 1. Growth of lens cells on posterior capsular bag. The lens epithelial cells proliferate starting at the outer edge and migrate in. The cells at very high density cause wrinkling of the capsule, while the cells in the middle are at a lower density.

FIG. 2. Fluorescent studies of porcine lens epithelial (PLE) cells: All conditions were incubated with a fluorescein isothiocyanate (FITC) labeled goat anti-rabbit IgG antibody as a second antibody. A. PLE cells stained with a non-specific rabbit antibody shows minimal staining. B. PLE cells stained with the rabbit anti-α B crystallin antibody shows increased staining. C. Hi magnification with rabbit anti-α B crystailin antibody shows localization of the stain in the cytoplasm.

FIG. 3: Western blot of PLE cells on a nitrocellulose membrane from a 15% sodium dodecyl sulfate-polyacrylamide gel electrophoresis (SDS PAGE), showing anti-α B crystallin antibody staining: Lanes 1&2. 20 μg of the soluble fraction of homogenized pig lens, +and−anti-α B crystallin antibody. Lane 3. 10μg purified porcine a crystallin +anti-α B crystallin antibody. Lanes 4&5. 50μg of the soluble fraction of homogenized PLE cells +and −anti α B crystallin antibody. In lane 1 the arrows indicate two bands. The top band shows strong specific staining of the antibody indicating that it is the a B subunit. This band is also present in the PLE cells, (lane 4). The lower band that stains weakly is strongly present in the protein stain (not shown), of the soluble fraction of the lens homogenate and also in the purified a crystallin, indicating that it is the α A crystallin subunit. The non-specific band in lane 5 of the PLE cells is at the level of the lower arrow, while there is no staining at the level of the α B crystallin.

FIG. 4. PLE Cell Growth: Cells were plated as in the toxicity study. Media was changed at day 1. Cells were counted every 24 hours, using 6 wells in duplicate.

FIG. 5. Toxicity of Picolinic Acid and its analogs in Pig Lens Epithelial Cells. Pig lens epithelial cells were plated in quadruplicate in 24 well trays at 5000 cells/well using minimal essential media plus 10% fetal calf serum (10% FCS MEM) with antibiotics. At 24 hours after plating, substances were added with a media change, at appropriate concentrations. Cells were allowed to grow for an additional 120 hours in the presence of the various substances. Cells were then counted, four dishes, in duplicate, and the cell counts were expressed as a percentage of the control cell counts.

FIG. 6. Induction of Apoptosis in PLE cells. PLE cells were grown in 35 mm dishes in tissue culture. Analogs were added to media 24 hours after cells were plated. After 48 hours of incubation, floating or supernatant cells were spun onto slides and stained using the DNA fragmentation kit. The conditions are as follows: A. Control monolayer; B. Control stained supernatant; C. 3 mM picolinic acid monolayer; D. 3 mM picolinic acid stained supernatant; E. 0.3 mM fusaric acid monolayer; F. 0.3 mM fusaric acid stained supernatant. At this time and concentration, picolinic acid inhibits growth of the monolayer with few floating cells while fusaric acid shows many floating cells, with strong nuclear staining. By 72 hours, the picolinic acid also exhibited many floating cells (not shown), while the control showed few floating cells at both 48 and 72 hours. There was some staining in floating cells in the control.

### DETAILED DESCRIPTION OF THE INVENTION

The present invention provides compositions capable of inhibiting the growth of lens epithelial (LE) cells. The invention also provides pharmaceutical compositions capable of inhibiting the growth of LE cells made up of at least one agent capable of inhibiting LE cell growth in combination with one or more excipients which are acceptable for topical application to the eye.

In a preferred embodiment, the agent for inhibiting LE cell growth is picolinic acid or an analog thereof. Especially preferred analogs of PA are those with a pyridine ring moiety, as shown in formula I. Picolinic acid (PA), as shown in formula II is a natural metabolite (59). Many of its biological activities are intrinsically tied to its ability to chelate trace metals. For example, Zn-picolinate has been shown to facilitate Zn2+absorption across the intestine and thus reverse zinc deficiencies (60). Conversely, the ability of picolinic acid to block cell division has been attributed to its ability to interfere with the function of zinc finger and iron binding proteins and their strategic involvement in DNA replication (61).

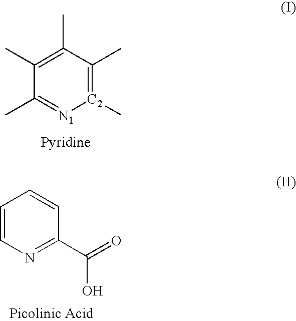

Without wishing to be bound by theory, PA and its analogs may be able to inhibit LE cell growth through their interaction with the protein transferrin (Tf). McGahan (62) has demonstrated that Tf, the plasma iron transport protein, is produced by LE cells, but not by iris or ciliary epithelium or corneal endothelium. Davidson et al. (63) found that Tf secretion was 5-fold higher in the after-cataract capsular bag containing regenerated lens tissue than in the contra lateral controls, indicating that the Tf was newly produced by proliferating LE cells. In tissue culture, cell survival rate decreased when the media was changed daily, as compared to the survival rate when the media was not changed, suggesting secretion of essential growth factors by the proliferating LE cells. Based on this study, LE cell proliferation was inhibited by an anti-Tf monoclonal antibody (64). The significant role of Tf in proliferating lens epithelial cells and the role of Zn in cell division could render these cells susceptible to the action of chelators at concentrations which the other eye tissues could tolerate.

PA and its analogs are chelators of trace metals including iron and zinc. Consequently, Tf should be one of the proteins that is affected by these agents. It has been shown that after the removal of PA from tissue culture medium in cells that have been synchronized in G1 growth phase, there is an increased uptake of ⁵⁹Fe-transferrin, and that the effect of PA on the normal rat kidney epithelial (NRK) cells is primarily caused by iron deprivation to the cells (65). This chelating ability of PA enables it to compete with zinc and iron binding proteins for the available zinc and iron. Consequently, at appropriate concentrations, PA can disrupt the biological activity of these metal binding proteins particularly in actively dividing or transformed cells. As shown in the Examples below, PA can inhibit the proliferation of LE cells in vitro.

Non-limiting examples of PA analogs contemplated by the invention include fusaric acid (formula III), di-piconlinic acid (formula IV), nicotinic acid (formula V). It is still further contemplated that other agents capable of inhibiting LE cell growth may be used in other embodiments of the present invention, including other chelating agents. Non-limiting examples of other chelating agents include dendrimers that are amino as well as carboxy terminated, such as those described by Boas et al. (86), Svenson et al. (87) and Lee et al. (88), and crown ethers such as those described by Takagi et al. (89). It is also contemplated that more than one agent may be used in combination in the compositions of the present invention.

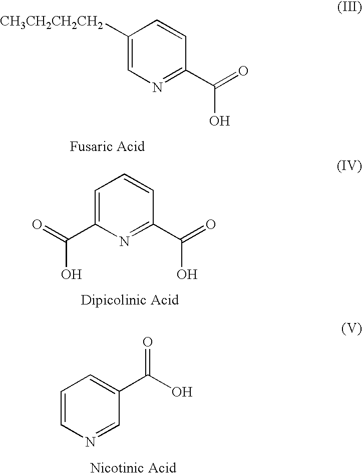

Various types of compositions containing an agent capable of inhibiting LE cell growth are contemplated by the invention. Preferably, the compositions of the invention are pharmaceutical compositions that may be applied topically to the eye. Other, non-pharmaceutical, compositions containing an agent capable of inhibiting LE cell growth are also contemplated by the invention. These non-pharmaceutical compositions may include various carriers that are able to dilute or dissolve the agent. Non-limiting examples of carriers include aqueous solvents, organic solvents and various polymers and salts. These non-pharmaceutical compositions may be used for in vitro treatment of LE cells, such as in a laboratory setting.

Various excipients are contemplated for formulation in the pharmaceutical compositions of the present invention. Various isotonic compounds, buffers, preservatives and pH adjusting compounds may be used as excipients in the present invention. Non-limiting examples of excipients which may be used in the compositions of the invention include: water, benzalkonium chloride, sodium edetate, polyvinyl alcohol, methylparaben, boric acid and cyclodextrins. It should also be apparent that there are other compounds not listed which are suitable for topical application to the eye that can be used within the scope of the present invention.

The pharmaceutical compositions of the present invention are meant to be applied topically to the eye or the to tissues surrounding the eye. In a preferred embodiment, the pharmaceutical compositions of the invention are formulated so that they may be administered drop wise into the eye. It is also contemplated that the pharmaceutical compositions of the invention may be formulated so that they may be sprayed into the eye or applied directly to the eye by other means.

Preferably, the pharmaceutical compositions of the present invention are used for the treatment and prevention of presbyopia. When used for the treatment of presbyopia, the pharmaceutical compositions of the present invention are applied as often as necessary in an amount sufficient to treat presbyopia until the symptoms of presbyopia diminish or disappear. When used for the prevention of presbyopia, the pharmaceutical compositions of the present invention are applied as often as necessary in an amount sufficient to treat presbyopia for as long as the patient wishes or for a length of time suggested by a medical professional. As the active agents of the pharmaceutical compositions of the present invention are preferably natural metabolites, they can be administered over a period of years, decades or even a lifetime. It is also contemplated that the pharmaceutical compositions of the present invention may be used for the prevention and treatment of posterior capsular opacification (PCO). It is still further contemplated that the pharmaceutical compositions of the present invention may be used for the treatment of other disorders whose pathologies are related to the growth of LE cells.

It is further contemplated that the pharmaceutical compositions of the present invention may be used in combination with other treatments of the eye. These treatments may include other medicinal treatments, such the topical or oral administration of other pharmaceutical agents. These treatments may also include surgical procedures, including surgical procedures done with lasers. As a non-limiting example, the pharmaceutical compositions of the present invention may be administered in combination with the surgical procedure described by Aliyar et al. (80), wherein administration of the pharmaceutical compositions of the present invention both pre- and post-surgery is contemplated.

Preferably, the compositions and methods of the present invention inhibit the growth of LE cells by killing all or part of the LE cells. One contemplated mechanism by which the compositions and methods of the present invention kill LE cells is by causing the cells to undergo apoptosis. It is also contemplated that the compositions and methods of the present invention may only stop or slow the growth of the LE cells without causing their death. Thus, an inhibition of the growth of LE cells is any effect whereby the LE cells grow more slowly than they would have if they were untreated, up to, and including, causing the death of 100% of the LE cells present.

It should be apparent to those skilled in the art that there are other embodiments of the present invention not specifically set forth herein that fall within the scope and the spirit of the invention as set forth in the claims below.

### EXAMPLES

### Example 1

### Materials and Methods

Porcine eyes were procured from a local slaughterhouse. All tissue culture products, PA and its analogs were purchased from Sigma-Aldrich (St. Louis, Mo.). The rabbit anti-αB-crystallin antibody was purchased from Biodesign International (Saco, ME); the goat anti-rabbit immunoglobulin G (IgG) antibody from Vector Labs (Burlingame, CA); and the DNA fragmentation detection kit from Oncogene Research Products (Boston, Mass.).

Isolation of Porcine Lens Epithelial Cells

The porcine eyes, obtained within 2 hours of sacrifice, were dissected under sterile conditions. The cornea was excised, and the anterior capsule was punched with an 8.00 mm trephine and gently removed with forceps. The contents of the capsular bag were easily prolapsed, and the posterior portion of the lens capsule was left attached to the sclera by the ciliary body and zonules. The remainder of the sclera, below the plane of the posterior lens, and vitreous were cut away. The attached lens capsule was then submersed in minimal Eagle medium (MEM) that contained 10% fetal calf serum (FCS) with antibiotics (gentamycin, penicillin, streptomycin, and amphotericin B), to allow attached lens epithelial cells to multiply. This medium was used for all of the subsequent experiments and was changed every three days until the cells were confluent on the posterior capsule. This method is a modification of procedures done by other groups (81). The cells were then trypsinized, and the resulting detached cells were collected and transferred to tissue culture flasks. The cells were grown and characterized.

Kim et al. (82) have done a definitive study of gene expression in a system similar to the one used. A Western blot and immunofluorescence were used to test for the presence of αB-crystallin protein. For immunofluorescence, PLE cells were plated on sterile cover slips that were placed in 60-mm tissue culture dishes. Cells were grown to 40% to 50% of confluence, with a medium change every 48 hours. Subsequently, cells were fixed in 4% formalin in phosphate-buffered saline (PBS) for 15 minutes and dehydrated with 50% methanol for two minutes, followed by 100% methanol for two minutes. After the methanol was drained, the dishes were stored at −20° C. until the cells were stained. For staining preparation, cover slips with cells were rinsed for two minutes in 100% methanol and for two minutes in 50% methanol and were then placed in water. The cells were rinsed three times with PBS after each step and incubated at room temperature. Subsequently, they were made permeable in 0.05% saponin for 30 minutes and then incubated with a blocking solution that contained 10% normal goat serum in PBS for 20 minutes. The blocking solution was removed, and a 1:100 dilution of rabbit antiαB-crystallin antibody was added in 1% bovine serum albumin (BSA)/PBS for 60 minutes. Cells were rinsed and incubated with a goat anti-rabbit IgG antibody with a label of fluorescein isothiocyanate (FITC) (15 μg/mL) in 1% BSA/PBS. Cells were rinsed and mounted on slides for viewing and photography.

To further confirm the presence of αB-crystallin, a sodium dodecyl sulfate-polyacrylamide gel electrophoresis (SDS-PAGE), (83) was performed followed by a Western blot analysis. The following three samples were prepared: (I) Total lens crystallins were obtained by homogenizing decapsulated porcine lenses for 30 strokes in a loose-fitting homogenizer. The sample was centrifuged at 10,000 g for 1 hour, and the supernatant was collected. (2) Purified α-crystallins were obtained by gel filtration chromatography. The total lens crystallins were applied to a 5-×100-cm Sephacryl 300 column (Pharmacia, Piscataway N.J.) and were fractionated. The α-crystallin fraction was collected. (3) For electrophoresis, PLE cells were grown in 150-mm dishes to 80% confluence. Cells were collected, homogenized, and centrifuged at 10,000 g for 15 minutes. The supernatant was obtained. Depending on the sample, 10 to 50 μg of protein was added per lane.

Samples were solubilized in a buffer that contained 1% SDS, 50 mmol/L Tris pH 7.4, 4.5 mmol/L EDTA, 10 mmol/L dithiothreitol, 1 mmol/L phenylmethylsulfonyl fluoride and underwent electrophoresis in a 15% polyacrylamide gel. Proteins were blotted onto a 0.45 μm nitrocellulose membrane (BioRad, Hercules, Calif.) overnight at 15 V. A Western blot analysis was performed with a rabbit polyclonal antiαB-crystallin antibody at a 1:100 dilution, and the proteins were detected with a Western blot analysis detection kit from Vector Labs. This kit includes a biotinylated goat anti-rabbit IgG second antibody, with a streptavidin alkaline phosphatase, and a chromogenic detection system with a nitroblue tetrazolium chloride and 5-bromo-4-cholor-3-indoylphosphate p-toluidine salt.

Determining 50% Inhibitory Concentration

Growth curve data were obtained from cells plated without any agents. Cell counts were performed daily on a total of eight wells per point, by combining four wells per count and performing counts in duplicate.

The toxicity of PA and its analogs to the PLE cells was tested according to the method of Senderoff et al. (66) PLE cells were plated in 24 well trays at 5000 cells per well in 1 mL of tissue culture medium (see above). The medium was changed at 24 hours, and new medium containing the analogs was added. Five days after addition of substances, cell counts were performed using a total of four wells per point, by combining two wells per count, and perfoming counts in duplicate. Results were expressed as a percentage of the untreated or control cell counts. From plots of the percentage cell count versus concentration, the IC50 (i.e., the concentration at which treated cell counts are 50% of the control) was established, and this was used as a measure of toxicity.

Apoptosis

The ability of PA and its analogs to induce apoptosis was detected with the DNA fragmention detection kit (Oncogene Research Products, Boston, Mass.). One of the molecular effects of apoptosis is the formation of oligonucleosomal DNA fragments by DNA laddering (67). The kit includes a Klenow DNA polymerase that labels 3′-OH terminals of DNA, which are formed in the laddering process. Incorporated biotinylated nucleotides are then detected with a chromogenic labeling system of streptavidin-horseradish peroxidase diaminobenzidine. Cells were plated in 35-mm wells at a density of 50,000 cells per well. 24 hours after plating, analogs were added concurrently with fresh media. Pictures were taken after 48 hours of exposure to the analogs. Floating cells were washed and placed in Saccomanno's fixative (Shandon, Pittsburgh, Pa.), centrifuged onto Superfrost slides (Fisher Scientific, Pittsburgh, Pa.) with a CytoSpin centrifuge (Shandon, Pittsburgh, Pa.), and stained according to the protocol given.

Testing of Toxicity in Corneal endothelium: To test for the effects of PA and FA on corneal endothelium, fresh corneas from pig eyes were aseptically dissected out, with a 2 mm scleral ring and placed in 2% FCS M-199 medium with antibiotics, as M-199 medium has previously been used for corneal experiments (84).

Times of incubation and concentrations which were used in the apoptosis experiment were implemented, i.e. 48 hours at 3 mM PA and 0.3 mM FA. Serum concentration of 2% was employed to maintain the corneal cells without undo stimulation. Corneas were rinsed with Hanks Balanced Salt Solution (HBSS) and stained for 90 seconds with 0.2 mL 1% Janus Green (85) in HBSS as a test for viability. The corneas were again rinsed. Negative controls were set up by placing corneas in 100% ethanol for 5 minutes prior to addition of Janus Green, to give staining in 100% “damaged” corneas. Using an 8 mm trephine, the central section of the cornea was punched out, and the dye was eluted with 2 ml 100% ethanol and the absorbance was measured at 650 nm. The Janus Green staining experiment of corneas was repeated twice. Duplicate samples were used for drug conditions and triplicate samples for controls. Janus green staining was also carried out on fresh cornea, for comparison purposes.

### Example 2

### Structures and Properties of PA Analogs

First, the PA analogues were constructed on a personal computer using the model-builder tool box within HyperChem software (Hypercube Inc., Gainesville, Fla.). Then, PM3, semi-empirical method, was used to identify the geometry of each compound at its lowest energy for the neutral, ground state structures. The settings for this procedure were as follows: a spin multiplicity of 1. a restricted Hartree-Fock method for spin pairing, a no-configuration interaction, a self-consistent field (SCF) convergence limit of 0.1, and a maximum iteration limit of 50 without accelerated convergence. The Polak-Ribiere conjugate-gradient algorithm, a geometry optimizing procedure, terminated when the Root-Mean Square (RMS) gradient reached 0.005 kcal/A mol). After optimization, the partial charges were calculated on nitrogen and the adjacent carbon C2, containing the carboxylic group to examine the charge distribution of the structures. Mulliken's method was used to calculate partial charges in Molecular Orbital Theory. The QSAR program from the CHEMPLUS subroutine was then used to acquire LogP (the log of the partition coefficient for the compound between octanol and water), volume, surface area, hydration energy, refractivity, polarizability, and mass.

### Example 3

### Growth of PLE cells

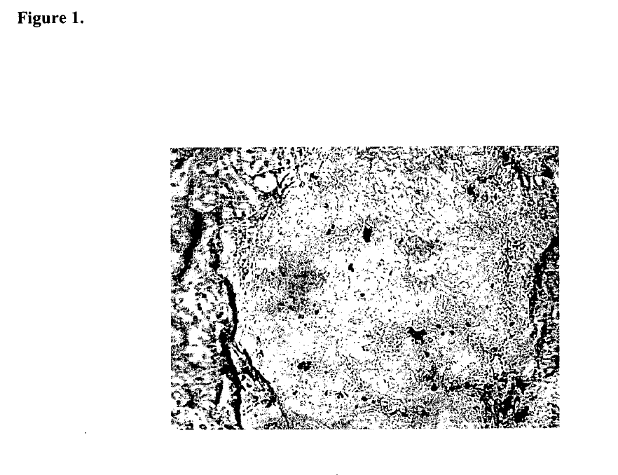

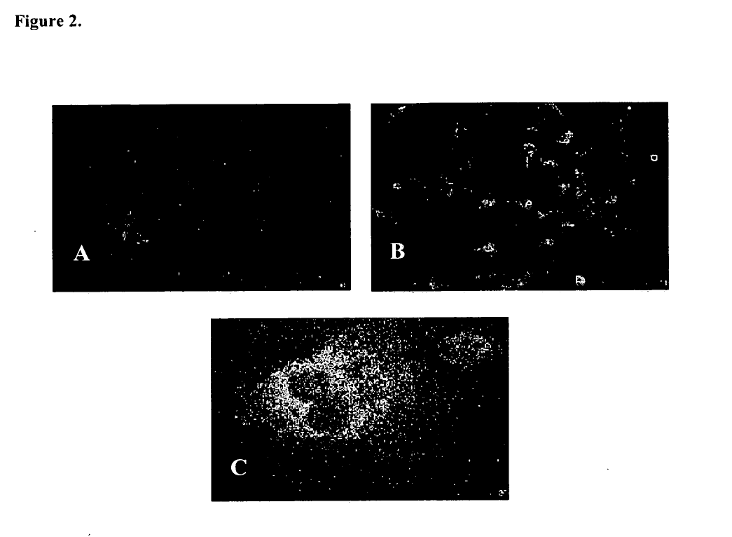

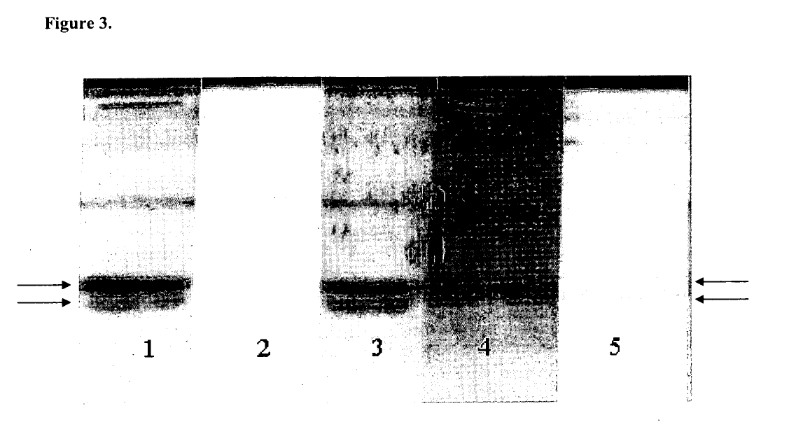

The PLE cells grew from the periphery of the lens capsule margin and took about 2 weeks to become confluent (FIG. 1) on the posterior lens capsule in 10% FCS/MEM. The extent of wrinkling of the posterior capsule was a function of the density of the cells. In general, the equatorial regions had a greater density of epithelial cells and exhibited greater wrinkling compared with the center of the posterior capsule. Fluorescent staining with a rabbit polyclonal primary antibody against αB-crystallin specifically stained PLE cells. Examination at high magnification showed the staining to be localized in the cytoplasm (FIG. 2a, 2b). Further evidence that the PLE cells were showing the αB-crystallin was confirmed by the presence of a low molecular weight band in SDS-PAGE, which co-migrated with purified α-crystallins, and was detected through the staining in Western blot analyses with the rabbit antiαB-crystallin antibody (FIG. 3). This band was absent in PLE cells stained in Western blot analysis, with the omission of primary antibody. There were some bands at higher molecular weights, probably due to non-specific staining.

### Example 4

### Determination of Toxicity of PA and its Analogs to PLE Cell Growth

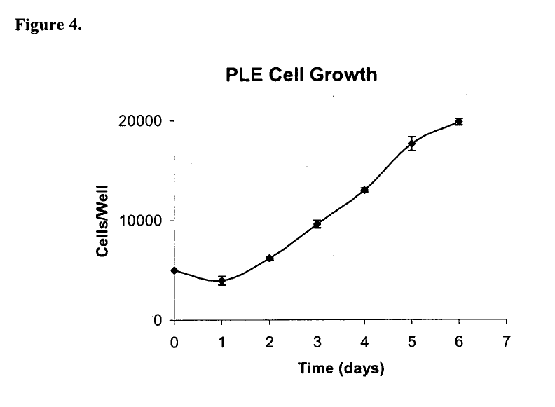

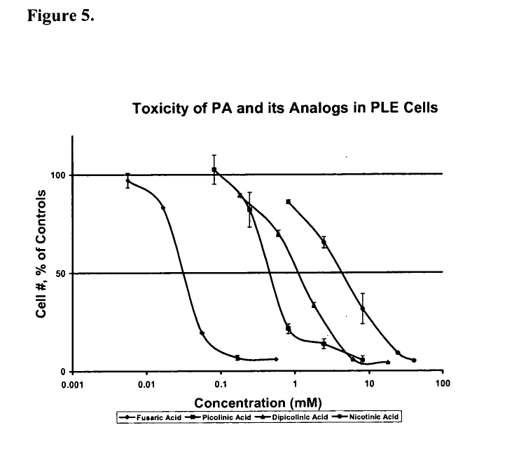

The growth curve of the PLE cells was characterized by a day of lag phase, followed by logarithmic growth (FIG. 4), and indicated a doubling time of about 48 hours for this cell line. From the IC50 data, it was observed that PA and its analogs exhibited a wide variation in toxicity (FIG. 5). As seen in Table 1, fusaric acid was the most toxic, about 15 times more toxic (on concentration basis) than PA, whereas nicotinic acid was about 10 times less toxic than PA. Dipicolinic acid was 2.5 times less toxic than PA.

TABLE 1
Toxicity Concentrations of Picolinic Acid and Its Analogs*

| Analog | IC₅₀ μg/mL | IC₅₀ mmol/L |
|---|---|---|
| Fusaric acid | 5.6 | 0.031 |
| Picolinic acid | 55.3 | 0.449 |
| Dipicolinic acid | 185 | 1.107 |
| Nicotinic acid | 539 | 4.378 |

*The concentrations are from FIG. 5. The cell count in the treated conditions is equal to 50% of the control cell count. This is then used to compare the relative toxicity of PA with its analogs.

### Example 5

### The Role of Apoptosis in PLE Cell Toxicity

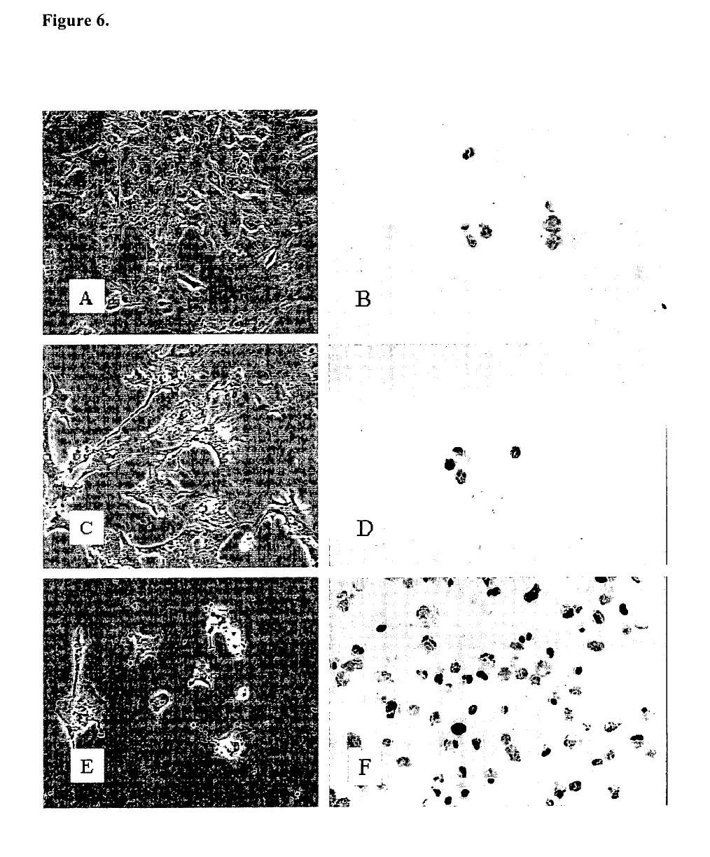

To test for the role of apoptosis in the toxicity of PA and its analogs, the PLE cells were exposed to 0.3 mmol/L FA and 3 mmol/L of PA. In FIG. 6, when comparing the monolayer of control (A), to PA treated (C) and FA treated (E) PLE cells, both morphology and cell density of the monolayer are markedly affected. When the cells in the supernatant of the corresponding monolayers were stained to detect DNA laddering, which is indicative of apoptosis (67), staining was detected in their nuclei. In the medium of control cells (B), there were only a few cells, and staining was not very intense. In medium of that contained PA there was also only a few floating cells present at 48 hours, but the staining was more intense. In medium that contained FA, there were many more floating cells and also the staining was intense. These results indicate in the PA treated cells, at this stage there is inhibition of cell growth but little cell death, while in the FA condition there is most likely inhibition of growth, but there is also significant cell death by the apoptotic pathway. Additional dishes of cells were maintained up to 72 hours, (results not shown) at which time no living cells were seen in the FA condition, very few cells were still attached in the PA condition, while the control cell monolayer was confluent.

In the Janus Green staining of the cornea, the control cornea which were fixed in methanol produced a darkly stained endothelial layer, while fresh corneal endothelial cells stained very little. Corneal endothelium maintained in the 2% FCS M199 medium for 48 hours actually stained a significant amount, as seen by the values in Table 2. Corresponding cornea treated with either PA or FA actually stained less than the untreated control, as demonstrated by the values of the eluted stain.

TABLE 2
Janus Green absorbance values of stain eluted
from trephine cut-outs of treated Corneas.

| Condition | Avg Abs₆₅₀/SD |
|---|---|
| Cont, 48 hr | 0.445 ± 0.116 |
| EtOH Cont 48 hr | 0.612 ± 0.087 |
| PA 3 mM 48 hr | 0.321 ± 0.041 |
| FA 0.3 mM 48 hr | 0.287 ± 0.011 |
| Cont fresh eyes | 0.098 ± 0.035 |
| EtOH control fresh eyes | 0.765 ± 0.179 |

Discussion

As mentioned above, there is an interest in identifying natural agents that can inhibit lens epithelial growth as a possible treatment for presbyopia as well as PCO. In previous investigations, Chinese hamster ovarian (CHO) cells had been used to investigate the effect of PA analogues on growth. Although insightful information was obtained, the CHO cell line is a transformed one in which PA has been shown to have effect. Consequently, it was important to show that these effects could be also be observed in untransformed normal lens epithelial cells. To obtain cells, which would be suitable for this experiment, a cell line was needed, which resembled human native lens epithelium, and yet could be maintained in tissue culture and grew more rapidly than human lens epithelial cells in tissue culture. The growth of human lens epithelial cells in vitro has been well documented, (68-71). It is difficult to use genetically unmodified human LE cell lines because in general, untransformed human lens epithelial cell lines have a limited growth capacity, typically of 5-7 population doublings and slow growth rates. It has been previously noted that bovine and rabbit lens epithelial cells do withstand multiple passages in tissue culture; however, as porcine lenses were already being used to investigate lens viscoelasticity, it was more meaningful to extend this investigation using porcine lens. It was found that PLE can be grown in tissue culture medium, and therefore cells obtained directly from the pig lens were chosen. Cells in tissue culture lose their identity after numerous passages and the same is the case for immortalized lens epithelial cells as well as cells in posterior capsular opacification (PCO).

Consequently, it was critical for this investigation, that it be confirmed that the PLE cells continued to express crystallins in spite of losing their fiber like morphology. This PLE cell line did continue to maintain its expression of α-B crystallins in tissue culture.

Toxicity of PA and its analogs exhibit varying degrees of efficacies which may depend mainly on two factors: the analog's ability to chelate trace metals like iron and zinc, and its ability to cross cell membranes. For example, nicotinic acid has a low toxicity compared to PA. Our calculations, based on semi-empirical quantum mechanics, showed that the oxygen on the carboxyl group is too far from the nitrogen for stable chelation with trace metals. Fusaric acid has a high toxicity because while having a same chelating ability as picolinic acid, the addition of the butyl group at the 5 position increases its logP (hydrophobicity) and thus its ability to cross cell membranes.

Furthermore, zinc has been shown to play a central role in apoptosis, (72;73), in that Ca2+ and Mg2+ dependent endonuclease activity in isolated nuclei was inhibited when Zn2+ was added to the medium, giving rise to a hypothesis that Zn2+ prevents apoptosis by blocking the activation of Ca2+-and Mg2+ dependent endonucleases. Similarly, the addition of PA and its analogs has been shown to induce apoptosis in many cell lines, especially cancerous cell lines, (74;75). Recently, Geissler (76;77) has shown that cacimycin exposure induced apoptosis in both rabbit and human LE cells. Jordon (78) has studied apoptosis in human LEC cells by the antimetabolic drug, mitomycin C. The experiments shown here indicate that fusaric acid and picolinic acid rapidly induced apoptosis in PLE cells. Unlike mitomycin C, PA and its analogs are significantly less toxic to quiescent cells. It has been shown, for example that in WI-38 cells (79), a normal lung fibroblast cell line, and confluent cells could withstand 48 hour incubation in 0.5 mM fusaric acid with no significant drop in viability. However, logarithmically growing WI-38 cells showed a decrease in viability of 76% by 30 hours. As shown, picolinic acid and its analog fusaric acid were significantly toxic to our PLE cell line and may have activated the apoptosis pathway. The next question which needs to be addressed in the study of these materials for the treatment of PCO or presbyopia is to determine their toxicity to the corneal endothelium and surrounding tissues. It is possible that there could be a selective toxicity due to the proliferative nature of PLE cells. Picolinic acid and its analogs need to be tested on whole lenses ex-vivo, as well as in a sustained delivery system or a onetime application by use in irrigating solutions during extra capsular lens extraction. While it remains necessary to test these analogs in vivo, these analogs do hold promise and have several desirable characteristics for in vivo use.

While a preferred embodiment and various uses have been set forth above, those skilled in the art who have reviewed the present disclosure will readily appreciate that other embodiments can be realized within the scope of the invention. For example, disclosures of the type and source of cells, the concentrations used and the effects of the various agents on cell growth are illustrative rather than limiting. The disclosure is not meant to be limited to use with LE cells from a specific organism, and use of the compositions and methods of the present invention with human LE cells is contemplated. Moreover, the present invention is applicable to other compositions in various forms that may be used for the inhibition of LE cell growth. Therefore, the present invention should be construed as limited only by the appended claims.

### REFERENCES

1. Boulton M and Saxby L. Basic Science of the Lens. Yanoff, M and Duker, J. S. Ophthalmology. 2nd ed. St. Louis: Mosby; 2004. pp. 241-5.
2. Kuszak J R and Brown H G. Embryology and Anatomy of the Lens. Albert, D. M. and Jakobiec, F. A. Principles and Practices of Ophthalmology. Basic Sciences. 1st ed. Philadelphia: W. B. B. Saunders; 1994. pp. 82-96.
3. Weale R A. Why We Need Reading-Glasses Before a Zimmer-Frame. Vision Res.; 40(17):2233-40.
4. Pierscionek B K and Weale R A. Risk Factors and Ocular Senescence. Gerontology 1996;42(5):257-69.
5. Marcantonio J M and Vrensen G F. Cell Biology of Posterior Capsular Opacification. Eye 1999:13 (Pt 3b):484-8.
6. Spalton D J. Posterior Capsular Opacification After Cataract Surgery. Eye 1999;13 (Pt 3b):489-92.
7. Marcantonio J M and Vrensen G F. Cell Biology of Posterior Capsular Opacification. Eye 1999:13 (Pt 3b):484-8.
8. Frezzotti R and Caporossi A. Pathogenesis of Posterior Capsular Opacification. Part I. Epidemiological and Clinico-Statistical Data. J. Cataract Refract. Surg. 1990;16(3): 347-52.
9. Beers A P and Van der Heijde G L. In Vivo Determination of the Biomechanical Properties of the Component Elements of the Accommodation Mechanism. Vision Res. 1994;34(21)2897-905.
10. Glasser A, Croft M A, and Kaufman P L. Aging of the Human Crystalline Lens and Presbyopia. Int. Ophthalmol. Clin. 2001;41(2):1-15.
11. Coleman D J and Fish S K. Presbyopia, Accommodation, and the Mature Catenary. Ophthalmology 2001;108(9): 1544-51.
12. Weale R. Presbyopia Toward the End of, the 20th Century. Surv. Ophthalmol. 1989;34 (1)1:15-30.
13. Croft M A, Glasser A, and Kauftnan P L. Accommodation and Presbyopia. Int. Ophthalmol. Clin. 2001;41(2):33-46.
14. Glasser A and Campbell M C. Biometric, Optical and Physical Changes in the Isolated Human Crystalline Lens With Age in Relation to Presbyopia. Vision Res. 1999;39(11): 1991-2015.
15. Schachar R A. Cause and Treatment of Presbyopia With a Method for Increasing the Amplitude of Accommodation. Ann. Ophthalmol. 1992;24(12):445-7, 452.
16. Pointer J S. The Burgeoning Presbyopic Population: an Emerging 20th Century Phenomenon. Ophthalmic Physiol Opt. 1998; 18(4):325-34.
17. Weale R A. Epidemiology of Refractive Errors and Presbyopia. Surv. Ophthalmol. 2005; 48(5):515-43.
18. Weale R. Presbyopia Toward the End of the 20th Century. Surv. Ophthalmol. 1989;34(1): 15-30.
19. Cross W. Theory Behind Surgical Correction of Presbyopia. Ophthalmol. Clin. North Am. 2001;14(2):315-33, viii.
20. Qazi M A, Pepose J S, and Shuster J J. Implantation of Sclieral Expansion Band Segments for the Treatment of Presbyopia. Am. J. Ophthalmol. 2002; 134(6): 808-15.
21. Doane J F. Accommodating Intraocular Lenses. Curr. Opin. Ophthalmol. 2004; 15(1): 16-21.
22. Werner L, Pandey S K, Izak A M, Vargas L G, Trivedi R H, Apple D J, and Mamalis N. Capsular Bag Opacification After Experimental Implantation of a New Accommodating Intraocular Lens in Rabbit Eyes. J. Cataract Refract. Surg. 2004;30(5): 1114-23.
23. Rana A, Miller D, and Magnante P. Understanding the Accommodating Intraocular Lens. J. Cataract Refract. Surg. 2003;29(12):2284-7.
24. Heatley C J, Spalton D J, Boyce J F, and Marshall J3. A Mathematical Model of Factors That Influence the Performance of Accommodative Intraocular Lenses. Ophthalmic Physiol Opt. 2004;24(2):111-8.
25. Haefliger E and Parel J M. Accommodation of an Endocapsular Silicone Lens (Phaco-Ersatz) in the Aging Rhesus Monkey. J. Refract. Corneal Surg. 1994-10(5):550-5.
26. Koopmans S A, Terwee T, Barkhof J, Haitjema H J, and Kooijman A C. Polymer Refilling of Presbyopic Human Lenses in Vitro Restores the Ability to Undergo Accommodative Changes. Invest Ophthalmol. Vis. Sci. 2003;44(1):250-7.
27. Murthy S K and Ravi N. Hydrogels As Potential Probes for Investigating the Mechanism of Lenticular Presbyopia. Curr. Eye Res. 2001 ;22(5):384-93.
28. Spalton D J. Posterior Capsular Opacification After Cataract Surgery. Eye 1999; 13 (Pt 3b):489-92.
29. Spalton D J. Posterior Capsular Opacification After Cataract Surgery. Eye 1999; 13 (Pt 3b):489-92.
30. Frezzotti R and Caporossi A. Pathogenesis of Posterior Capsular Opacification. Part I. Epidemiological and Clinico-Statistical Data. J. Cataract Refract. Surg. 1990;16(3):347-52
31. Frezzotti R, Caporossi A, Mastrangelo D, Hadjistilianou T, Tosi P, Cintorino M, and Minacc i C. Pathogenesis of Posterior Capsular Opacification. Part II: Histopathological and in Vitro Culture Findings. J. Cataract Refract. Surg. 1990; 16(3):353-60.
32. Marcantonio J M and Vrensen G F. Cell Biology of Posterior Capsular Opacification. Eye 1999.13 (Pt 3b):484-8.
33. Spalton D J. Posterior Capsular Opacification After Cataract Surgery. Eye 1999;13 (Pt 3b):439-92.
34. Charles S. Vitreoretinal Complications of YAG Laser Capsulotomy. Ophthalmol. Clin. North Am. 2001; 14(4):705-10.
35. Jahn C E, Richter J, Jahn A H, Kremer G, and Kron M. Pseudophakic Retinal Detachment After Uneventful Phacoemulsification and Subsequent Neodymium: YAG Capsulotomy for Capsule Opacification. J. Cataract Refract. Surg. 2003;29(5):925-9.
36. Nishi O. Nishi K, Saitoh I, and Salcanishi K. Inhibition of Migrating Lens Epithelial Cells by Sustained Release of Ethylenediaminetetraacetic Acid. J. Cataract Refract. Surg. 1996;22 Suppl 1:863-8.
37. Humphry R C, Davies E G, Jacob T J, and Thompson G M. The Human Abnterior Lens Capsule-an Attempted Chemical Debrodment of Epithelial Cells by Ethylenediaminetetracetic Acid (EDTA) and Trypsin. Br. J. Ophthalmol. 1988;72:406-8.
38. McDonnell P J, Krause W, and Glaser B M. In Vitro Inhibition of Lens Epithelial Cell Proliferation and Migration. Ophthalmic Surg. 1988;19(1):25-30.
39. Chung H S, Lim Si, and Kim H13. Effect of Mitomycin-C on Posterior Capsule Opacification in Rabbit Eyes. J. Cataract Refract. Surg. 2000;26(10):1537-42.
40. Ismail M M, Alio J L, and Ruiz Moreno J M. Prevention of Secondary Cataract by Antimitotic Drugs: Experimental Study. Ophthalmic Res. 1996;28(1):64-9.
41. You Y S, Lim S J, Chung H S, Lee Y G, Kim C Y, and Hong Y J. The Effective Concentration and Exposure Time of Mitomycin-C for the Inhibition of Lens Epithelial Cell Proliferation in Rabbit Eyes. Yonsei Med. J. 2000;41(2):185-9.
42. Haus C M and Galand A L. Mitomycin Against Posterior Capsular Opacification: an Experimental Study in Rabbits. Br. J. Ophthalmol. 1996;80(12):1087-91.
43. Power W J, Neylan D, and Collum L M. Daunomycin As an Inhibitor of Human Lens Epithelial Cell Proliferation in Culture. J. Cataract Refract. Surg. 1994;20(3):287-90.
44. Weller M, Wiedemann P. Fischbach R, Hartmann C, and Heimann K. Evaluation of Daunomyecin Toxicity on Lens Epitheliurn in Vitro. Int. Ophthalmol. 1988;12(2):127-30.
45. Pandey S K, Cochener B, Apple D J, Cohn J, Werner L, Bougaran R, Trivedi RH, Macky T A and Izak A M. Intracapsular Ring Sustained 5-Fluorouracil Delivery System for the Prevention of Posterior Capsule Opacification in Rabbits: a Histological Study. J. Cataract Refract. Surg. 2002;28(1): 139-48.
46. Ruiz J M, Medrano M, and Alio I L. Inhibition of Posterior Capsule Opacification by 5-Fluorouracil in Rabbits. Ophthalmic Res. 1990;22(4):201-8.
47. Pandey S K, Cochener B, Apple D J, Cohn J, Werner L, Bougaran R, Trivedi R H, Macky T A. and Izak A M. Intracapsular Ring Sustained 5-Fluorouracil Delivery System for the Prevention of Posterior Capsule Opacification in Rabbits: a Histological Study. J.Ctitaract Refract.Surg. 2002;28(1): L39-48.
48. Wu M, Li H, Li S, and Zeng J. The Effect of Liposome Encapsulated Daunorubicin on Rabbit Eyes After Extracapsular Lens Extraction. Yan. Ke. Xue. Bao. 2000;16(3): 189-93.
49. McDonnell P J, Krause W, and Glaser B M. In Vitro Inhibition of Lens Epithelial Cell Proliferation and Migration. Ophthalmic Surg. 1988;19(1):25-30.
50. Tetz M R, Ries M W, Lucas C, Stricker H, and Volcker H E. Inhibition of Posterior Capsule Opacification by an Intraocular-Lens-Bound Sustained Drug Delivery System: an Experimental Animal Study and Literature Review. J. Cataract Refract. Surg. 1996;22(8): 1070-8.
51. McDonnell P J, Krause W, and Glaser B M. In Vitro Inhibition of Lens Epithelial Cell Proliferation and Migration. Ophthalmic Surg. 1988;19(1):25-30.
52. McDonnell P J, Krause W, and Glaser B M. In Vitro Inhibition of Lens Epithelial Cell Proliferation and Migration. Ophthalmic Surg. 1988; 19(1):25-30.
53. Geissler P T, Li D W, and James E R. Inhibition of Lens Epithehial Cell Growth by Induction of Apoptosis: Potential for Prevention of Posterior Capsule Opacification. J. Ocul. Pharmacol. Ther, 2001;17(6):587-96.
54. Goins K M, Ortiz J R, Fulcher S F, Handa J T, Jaffe G J, Foulks G N, and Cobo L M. Inhibition of Proliferating Lens Epithehium With Antitransferrin Receptor Immunotoxin. J. Catartact Refract. Surg. 1994;20(5):5 13-6.
55. Nishi O, Nishi K, Wada K, Ohmoto Y, and Akura 3. Inhibition of Lens Epithelial Cells by Fas-Specific Antibody Activating Fas-Fas Ligand System. Curr. Eye Res. 2001 ;23(3): 192-8.
56. Tarsio J F, Kelleher P J, Tarsio M, Emery J M, and Lam D M. Inhibition of Cell Proliferation on Lens Capsules by 4197X-Ricin A Immunoconjugate. J. Cataract Refract. Surg. 1997;23(2):260-6.
57. Clark D S, Emery J M, and Munsell M F. Inhibition of Posterior Capsule Opacification With an Immunotoxin Specific for Lens Epithehial Cells: 24 Month Clinical Results. J. Cataract Refract. Surg. 1998;24(12): 1614-20.
58. Meacock W R, Spalton D J, Hollick E J, Boyce J F, Barman S, and Sanguinetti G. Double-Masked Prospective Ocular Safety Study of a Lens Epithehial Cell Antibody to Prevent Posterior Capsule Opaciftcaiion. J. Cataract Refract. Surg. 2000;26(5):716-21.
59. Mehler A L and May E L. Studies With Carboxyl-Labeled 3-Hydroxy-Anthranilic Acid and Picolinic Acids in Viva and in Vitro. J. Biol. Chem. 1956;223:449-55.
60. Evans G W and Johnson P E. Characterization and Quantitation of a Zinc-Binding Ligand in Human Milk. Pediatric Res. 1980;18:876-80.
61. Femandez-Pol.J A, Hamilton P D, and KIos D J. Essential Viral and Cellular Zinc and boo Containing Metalloproteins As Targets for Novel Antiviral and Anticancer Agenis: Implications for Prevention and Therapy of Viral Diseases and Cancer. Anticancer Res. 2001;21 (2A):931-57.
62. McGahan M C, Hamed J, and Grimes A M!. Transferrin Secretion by Lens Epithelial Cells in Culture. Exp. Eye Res. 1995;60:667-73.
63. Davidson M G, Hamed J, Grimes A M, Duncan G, Wormstone I M, and McGahan M C. Transferrin in After-Cataract and As a Survival Factor for Lens Epithelium. Exp. Eye Res. 1998;66(2):207-15.
64. Goins K M, Ortiz J R, Fulcher S F, Handa J T, Jaffe G J, Foulks G N, and Cobo L M. Inhibition of Proliferating Lens Epithelium With Antitransferrin Receptor Immunotoxin. J. Cataratct Refract. Surg. 1994;20(5):513-6.
65. Femandez-Pol J A, KIos D, and Donati R M. Iron Transport in NRK Cells Synchronized in G(1) by Picolinic Acid. Cell Biol. Int. Rep. 1978;2(5):433-9.
66. Senderoff R I, Weber P A, Smith D R, and Sokoloski T D. Evaluation of Antiproliferative Ageits Using a Cell-Culture Model; Invest Ophthalmol. Vis. Sci. 1990;31(12):2572-8.
67. Gavrieli Y, Sherman Y, and Ben-Sasson S A. Identification of Programmed Cell Death in Situ Via Specific Labeling of Nuclear DNA Fragmentation. J. Cell Biology 1992;1 19:493-501.
68. Liu C S, Wormstone I M, Duncan G, Marcantonio J M, Webb S F, and Davies P D. A Study of Human Lens Cell Growth in Vitro. A Model for Posterior Capsule Opacification. Invest Ophthalmol. Vis. Sci. 1996;37(5):906-14.
69. Jacob T I, Humphry R C, Davies E G, and Thompson G M. Cytological Factors Relating to posterior Capsule Opacification Following Cataract Surgery. Br. J. Ophthalmol. 1987;71 ( )):659.63.
70. El Osta A A, Spalton Di, and Marshall J. In Vitro Model for the Study of Human Posterior Capsule Opacification. J. Cataract Refract. Surg. 2003;29(8): 1593-600.
71. Rakic J M, Galand A, and Vrensen G F. Lens Epithelial Cell Proliferation in Human Posterior Capsule Opacification Specimens. Exp. Eye Res. 2000;71(5):489-94.
72. Sunderman F W, Jr. The Influence of Zinc on Apoptosis. Ann. Chin. Lab Sci. 1995;25(2): 134-42.
73. Fraker P J and Telford W G. A Reappraisal of the Role of Zinc in Life and Death Decisions of Cells. Zinc and Apoptosis. Proc. Soc. Exp. Biol. Med. 1997;215(3):229-36.
74. Ogata S. Inoue K, Iwata K, Okumura K, and Taguchi H. Apoptosis Induced by Picolinic Acid-Related Compounds in HL-60 Cells. Biosci.Biotechnol.Biochem. 2001; 65(10):2337-9.
75. Fernandez-Pol J A, Klos D J, and Hamilton P D. Antiviral, Cytotoxic and Apoptotic Activities of Picolinic Acid on Human Iminunodeficiency Virus-1 and Human Herpes Simplex Virus-2 Infected Cells. Anticancer Res. 2001;21(6A):3773-6.
76. Geissler F T, Li D W, and James E R. Inhibition of Lens Epithelial Cell Growth by Induction of Apoptosis: Potential for Prevention of Posterior Capsule Opacitication. J. Ocul. Pharmacol. Ther. 2001; h7(6):587-96.
77. Jordan J F, Kociok N, Grisanti S, Jacobi P C, Esser J M, Luther T T, Krieglstein G K, and Esser P. Specific Features of Apoptosis in Human Lens Epithelial Cells Induced by Mitomycin C in Vitro. Graefes Arch. Chin. Exp. Ophthalmol. 2001;239(8):613-8.
78. Jordan J F, Kociok N, Grisanti S, Jacobi P C, Esser J M, Luther T T, Krieglstein G K, and Esser P. Specific Features of Apoptosis in Human Lens Epithehial Cells Induced by Mitomycin C in Vitro. Graefes Arch. Clin. Exp. Ophthahnol. 2001;239(8):613-8.
79. Femandez-Pol J A, Klos D J, and Hamilton P D. Cytotoxic Activity of Fusaric Acid on Human Adenocarcinoma Cells in Tissue Culture. Anticancer Res. 1993;13(1):57-64.
80. Aliyar H A, Hamilton P D and Ravi N. Refilling of Ocular Lens Capsule with Copolymeric Hydrogel Containing Reversible Disulfide. Biomacromolecules 2005; 6:204-211.
81. Liu C S, Wormstone I M, Duncan G, Marcantonio J M, Webb S F, Davies P D. A study of human lens cell growth in vitro. A model for posterior capsule opacification. Invest Ophthalmol Vis Sci 1996; 37:906-914.
82. Kim J-T, Lee E H, Chung K-H, et al. Transdifferentiation of cultured lens epithelial cells into myofibroblast-like cells by serum modulation. Yonsei Med J 2004; 45:380-391.
83. Ausubel F, Brent R, Kingston R E, Moore D D, Seidman J G, Smith J A, Struhl K (eds). Current Protocols in Molecular Biololgy. Hoboken, N.J.: John Wiley and Sons; 1998; 4-10 (sec. 10.2), 1-8 (sec. 10.8).
84. Senoo T; Obara Y; Joyce N C. EDTA: a promoter of proliferation in human corneal endothelium. Invest Ophthalmol Vis Sci 2000; 41:2930-2935.
85. Hartmann C, Rieck P. A new test for endothelial viability. Arch Ophthalmol 1989; 107:1211-1215.
86. Boas U, Heegaard P M. Dendrimers in drug research. Chem Soc. Rev. 2004; 33(1):43-63.
87. Svenson S, Tomalia D A. Dendrimers in biomedical applications—reflections on the field. Adv. Drug Deliv. Res. 2005; 57(15)2106-29.
88. Lee C C, MacKay J A, Frechet J M, Szoka F C. Nat. Biotechnology 2005; 23(12):1517-26.
89. Takagi M, Nakano K, Nakashima N. Crown ethers as sensory materials in chemical measurements: Interfaces for chemical signal transformation. Pure & Appl. Chem. 1989:61(9):1605-1612.

What is claimed is:

1. A composition for inhibiting the growth of lens epithelial cells, comprising:
a chelating agent in an amount sufficient to inhibit lens epithelial cell growth; and
a carrier.

2. The composition for inhibiting the growth of lens epithelial cells of claim 1, wherein the chelating agent is picolinic acid.

3. The composition for inhibiting the growth of lens epithelial cells of claim 1, wherein the chelating agent is fusaric acid.

4. The composition for inhibiting the growth of lens epithelial cells of claim 1, wherein the chelating agent is di-picolinic acid.

5. The composition for inhibiting the growth of lens epithelial cells of claim 1, wherein the chelating agent is nicotinic acid.

6. The composition for inhibiting the growth of lens epithelial cells of claim 1, wherein the chelating agent is a dendrimer.

7. The composition for inhibiting the growth of lens epithelial cells of claim 1, wherein the chelating agent is a crown ether.

8. The composition for inhibiting the growth of lens epithelial cells of claim 1, wherein the carrier is an aqueous solvent.

9. The composition for inhibiting the growth of lens epithelial cells of claim 1, wherein the carrier is an organic solvent.

10. The composition for inhibiting the growth of lens epithelial cells of claim 1, wherein the carrier is a polymer.

11. The composition for inhibiting the growth of lens epithelial cells of claim 1, wherein the carrier is a salt.

12. A pharmaceutical composition for topical application to the eye, comprising:
a chelating agent in an amount sufficient to inhibit lens epithelial cell growth; and
one or more excipients.

13. The pharmaceutical composition for topical application to the eye of claim 12, wherein the chelating agent is picolinic acid.

14. The pharmaceutical composition for topical application to the eye of claim 12, wherein the chelating agent is fusaric acid.

15. The pharmaceutical composition for topical application to the eye of claim 12, wherein the chelating agent is di-picolinic acid.

16. The pharmaceutical composition for topical application to the eye of claim 12, wherein the chelating agent is nicotinic acid.

17. The pharmaceutical composition for topical application to the eye of claim 12, wherein the chelating agent is a dendrimer.

18. The pharmaceutical composition for topical application to the eye of claim 12, wherein the chelating agent is a crown ether.

19. The pharmaceutical composition for topical application to the eye of claim 12, wherein the one of more excipients are selected from the group consisting of: water, benzalkonium chloride, sodium edetate, polyvinyl alcohol, methylparaben, boric acid, cyclodextrins and combinations thereof.

20. The pharmaceutical composition for topical application to the eye of claim 12, wherein the topical application is for the purpose of treating or preventing a disorder of the eye.

21. The pharmaceutical composition for topical application to the eye of claim 20, wherein the disorder of the eye is presbyopia.

22. A pharmaceutical composition for the treatment or prevention of presbyopia, comprising:
a chelating agent in an amount sufficient to inhibit lens epithelial cell growth; and
one or more excipients.

23. The pharmaceutical composition for topical application to the eye of claim 22, wherein the one of more excipients are selected from the group consisting of:
water, benzalkonium chloride, sodium edetate, polyvinyl alcohol, methylparaben, boric acid, cyclodextrins and combinations thereof.

24. A method for inhibiting the growth of lens epithelial cells, comprising:
providing lens epithelial cells; and
contacting the lens epithelial cells with a composition comprising a chelating agent in an amount sufficient to inhibit lens epithelial cell growth.

25. The method for inhibiting the growth of lens epithelial cells of claim 24, wherein the chelating agent is picolinic acid.

26. The method for inhibiting the growth of lens epithelial cells of claim 24, wherein the chelating agent is fusaric acid.

27. The method for inhibiting the growth of lens epithelial cells of claim 24, wherein the chelating agent is di-picolinic acid.

28. The method for inhibiting the growth of lens epithelial cells of claim 24, wherein the chelating agent is nicotinic acid.

29. The method for inhibiting the growth of lens epithelial cells of claim 24, wherein the chelating agent is a dendrimer.

30. The method for inhibiting the growth of lens epithelial cells of claim 24, wherein the chelating agent is a crown ether.

31. A method for treating or preventing a disorder of an eye, comprising:
topically applying to the eye a composition comprising:
a chelating agent in an amount sufficient to inhibit lens epithelial cell growth; and
one or more excipients.

32. The method for treating or preventing a disorder of an eye of claim 31, wherein the chelating agent is picolinic acid.

33. The method for treating or preventing a disorder of an eye of claim 31, wherein the chelating agent is fusaric acid.

34. The method for treating or preventing a disorder of an eye of claim 31, wherein the chelating agent is di-picolinic acid.

35. The method for treating or preventing a disorder of an eye of claim 31, wherein the chelating agent is nicotinic acid.

36. The method for treating or preventing a disorder of an eye of claim 31, wherein the chelating agent is a dendrimer.

37. The method for treating or preventing a disorder of an eye of claim 31, wherein the chelating agent is a crown ether.

38. The method for treating or preventing a disorder of an eye of claim 31, wherein the disorder of the eye is presbyopia.

39. The method for treating or preventing a disorder of an eye of claim 31, wherein the one of more excipients are selected from the group consisting of:
water, benzalkonium chloride, sodium edetate, polyvinyl alcohol, methylparaben, boric acid, cyclodextrins and combinations thereof.
